(19) United States
(12) Patent Application Publication
(10) Pub. No.: US 2018/0104485 A1
(43) Pub. Date: Apr. 19, 2018

(54) DYNAMICALLY CONTROLLABLE APPARATUS FOR ASSISTING CARDIO-PULMONARY RESUSCITATION

(71) Applicant: MOR RESEARCH APPLlCATIONS LTD., Tel-Aviv (IL)

(72) Inventor: Nimrod ADI, Mazkeret Batya (IL)

(21) Appl. No.: 15/556,727

(22) PCT Filed: Mar. 7, 2016
(86) PCT No.: PCT/IL2016/050254

§ 371 Date: Sep. 8, 2017

Related U.S. Application Data
(60) Provisional application No. 62/129,999, filed on Mar. 9, 2015.

(51) Int. Cl. A61N 1/36; A61H 31/00; A61H 9/00; A61N 1/39; A61N 1/05; A61N 1/04

(52) U.S. Cl. CPC A61N 1/3601; A61H 31/006; A61H 9/0078; A61H 31/005; A61N 1/36031; A61H 2201/5002; A61N 1/0517; A61N 1/0456; A61H 2201/10; A61H 2201/5007; A61N 1/3987

(57) ABSTRACT

A cardiopulmonary resuscitation (CPR) assisting apparatus, comprising: a gasping-stimulating electrode configured to stimulate the thorax diaphragm to cause a gasping action; and a controller configured to control the stimulation applied by the gasping-stimulating electrode.

### TECHNICAL FIELD

The invention relates to the field of treating cardiac arrest.

### BACKGROUND

Sudden cardiac arrest, also known as sudden cardiopulmonary arrest, may result in insufficient perfusion of vital organs, which include the brain and the heart, eventually causing irreversible hypoxic damage to the organs.

Emergency treatment of a person suffering from cardiac arrest includes the employment of Cardio-Pulmonary Resuscitation (CPR) procedures, which may include external cardiac compression, defibrillation and/or cardiac pacing.

External cardiac compression is employed to maintain perfusion at a sufficient level, at least for some period of time, to provide arterial oxygenation and blood flow to the vital organs, thus increasing the chance to regain return of spontaneous circulation (ROSC) and, in turn, decrease the chance of brain and systemic damage. External cardiac compression is achieved by repetitively forcing the sternum against the heart to compress the heart between the sternum and the vertebral column and may be applied until spontaneous cardiac activity is re-established, e.g., through the above-mentioned defibrillation and/or cardiac pacing.

CPR may provide only about 10% to 30% of normal blood flow to the heart and only about 30% to 40% of normal blood flow to the brain. Accordingly, resuscitation rates of persons suffering out of hospital cardiac arrest (OHCA) are poor and range, e.g., between about 3% and 16%. The effectiveness of CPR is thus less than satisfactory.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in accordance with an embodiment, a cardiopulmonary resuscitation (CPR) assisting apparatus, comprising: a gasping-stimulating electrode configured to stimulate the thorax diaphragm to cause a gasping action; and a controller configured to control the stimulation applied by the gasping-stimulating electrode.

In some embodiments, the CPR assisting apparatus of further comprises a sensor configured to measure a physiological indicator and provide the measured indicator to the controller, wherein the controller is configured to control the stimulation responsive to receiving the measured indicator.

In some embodiments, the physiological indicator is an externally applied cardiac compression.

In some embodiments, the gasping-stimulating electrode comprises: an esophagus electrode configured to be positioned internal to the esophagus at the C6 vertebrate level; and a neck electrode configured to be positioned externally at a posterior border of the sternocliedomastoid muscles at the cricoid cartilage level, wherein the neck electrode is configured to transmit to the esophagus electrode a stimulating signal suitable for stimulating any of a cervical right and left phrenic nerves.

In some embodiments, the CPR assisting apparatus further comprises at least one expandable unit configured to be inserted into the esophagus; and an elongated body in fluid communication with the expandable unit and configured to allow a pressurized fluid to flow to and from the expandable unit, thereby allowing expandable unit to expand and contract, wherein the controller is configured to control the expansion and contraction of the expandable unit.

In some embodiments, the expandable unit comprises an expandable cardiac unit that is configured to render a portion of the esophagus substantially non-compliant.

In some embodiments, the expandable unit comprises an expandable descending-aorta unit that is configured to apply pressure on the descending aorta.

In some embodiments, the CPR assisting apparatus further comprises an expandable gastric unit is configured to apply pressure on a portion of the inner wall of the stomach.

In some embodiments, the sensor comprises a defibrillation/pacing electrode, wherein the controller is configured to control a defibrillating stimulus delivered to the heart by the defibrillation/pacing electrode.

In some embodiments, the defibrillation/pacing electrode is configured to be positioned internally and apply an internal defibrillation stimulus.

There is provided, in accordance with an embodiment, a cardiopulmonary resuscitation (CPR) assisting apparatus, comprising: at least one expandable unit configured to be inserted into the esophagus; an elongated body in fluid communication with the expandable unit and configured to allow a pressurized fluid to flow to and from the expandable unit, thereby allowing expandable unit to expand and contract; and a controller configured to control the expansion and contraction of the expandable unit.

In some embodiments, the expandable unit comprises an expandable descending-aorta unit that is configured to apply pressure on the descending aorta for increasing a venous return pressure gradient.

In some embodiments, the expandable unit comprises an expandable cardiac unit that is configured to render a portion of the esophagus substantially non-compliant and support to a posterior side of the heart during application of cardiac compression procedures.

In some embodiments, the expandable unit comprises an expandable gastric unit is configured to apply pressure on a portion of the inner wall of the stomach.

In some embodiments, the CPR assisting apparatus further comprises a sensor configured to measure a physiological indicator and provide the measured indicator to the controller, wherein the controller is configured to control the expansion and contraction responsive to receiving the measured indicator.

In some embodiments, the physiological indicator is an externally applied cardiac compression.

There is provided, in accordance with an embodiment, a method for assisting in the treatment of cardiac arrest, the method comprising: inserting at least one gasping-stimulating electrode into an esophagus; positioning the gasping-stimulating electrode in a position suitable for stimulating the thorax diaphragm; and delivering a signal to stimulate the thorax diaphragm to cause a gasping action.

In some embodiments, the method further comprises inserting at least one expandable unit into the esophagus; and positioning the expandable unit for increasing perfusion of vital organs by selectively setting the expandable unit in expanded or collapsed configuration.

There is provided, in accordance with an embodiment, a method for assisting in the treatment of cardiac arrest, the method comprising: inserting a first expandable unit into the esophagus; positioning the first expandable unit against at least a portion of the descending aorta; and expanding the first expandable unit to apply pressure to the descending aorta during the application of external cardiac compression.

In some embodiments, the method further comprises inserting a second expandable unit into the esophagus; positioning the second expandable to provide support to a posterior side of the heart; and expanding the second expandable unit to harden a portion of the esophagus, thereby providing support posterior side of the heart.

In some embodiments, the method further comprises inserting a second expandable unit into the esophagus; positioning the second expandable unit against the endothelial wall of stomach; and expanding the second expandable unit, thereby providing support to a lower ventricle of the heart.

There is provided, in accordance with an embodiment, a cardiopulmonary resuscitation (CPR) assisting apparatus, comprising: at least two expandable units configured to be inserted into the esophagus; an elongated body in fluid communication with the expandable units and configured to allow a pressurized fluid to independently flow to and from each of the expandable units, thereby allowing each expandable unit to independently expand and contract; and a controller configured to control the independent expansion and contraction of each of the expandable units.

In some embodiments, the at least two expandable units comprise any two of: an expandable descending-aorta unit that is configured to apply pressure on the descending aorta for increasing a venous return pressure gradient, an expandable cardiac unit that is configured to render a portion of the esophagus substantially non-compliant and support to a posterior side of the heart during application of cardiac compression procedures, and expandable gastric unit is configured to apply pressure on a portion of the inner wall of the stomach.

In some embodiments, the CPR assisting apparatus further comprises a third expandable unit comprising any of: an expandable descending-aorta unit that is configured to apply pressure on the descending aorta for increasing a venous return pressure gradient, an expandable cardiac unit that is configured to render a portion of the esophagus substantially non-compliant and support to a posterior side of the heart during application of cardiac compression procedures, and expandable gastric unit is configured to apply pressure on a portion of the inner wall of the stomach.

In some embodiments, the CPR assisting apparatus further comprises a sensor configured to measure a physiological indicator and provide the measured indicator to the controller, wherein the controller is configured to control the independent expansion and contraction of each of the expandable units responsive to receiving the measured indicator.

In some embodiments, the physiological indicator is an externally applied cardiac compression.

In some embodiments. The CPR assisting apparatus of claim 22, further comprises a gasping-stimulating electrode configured to stimulate the thorax diaphragm to cause a gasping action, wherein the controller is configured to control the stimulation applied by the gasping-stimulating electrode.

In addition to the exemplary aspects and embodiments described above, further aspects and embodiments will become apparent by reference to the figures and by study of the following detailed description.

### BRIEF DESCRIPTION OF THE DRAWINGS

The drawings and descriptions are meant to illuminate and clarify embodiments disclosed herein, and should not be considered limiting in any way.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. The figures are listed below.

FIG. 1 is an illustration of a CPR assisting apparatus, according to an embodiment;

FIG. 2A is a schematic sagittal side view illustration of the CPR assisting apparatus of FIG. 1 inserted into a human chest cavity, according to an embodiment;

FIG. 2B is a schematic illustration of a bilateral neck electrode in an operable position for triggering gasping in the patient, according to an embodiment;

FIG. 3 is an illustration of a CPR assisting apparatus, according to another embodiment;

FIG. 4A shows a flow chart of a method for using the apparatus shown in any of FIGS. 1, 2A-2B, and 3 to assist an application of CPR, according to an embodiment; and

FIG. 4B shows a flow chart of another method for using the apparatus shown in any of FIGS. 1, 2A-2B, and 3 to assist an application of CPR, according to an embodiment.

### DETAILED DESCRIPTION

Disclosed herein is an apparatus and methods for providing assistance when applying CPR to a patient suffering from cardiac arrest. According to an embodiment, the device may include any of: one or more expandable and collapsible units, stimulating electrodes, and sensors that are suitable for being orally inserted into a patient via the esophagus, or food-pipe. In an embodiment, based on a signal received from any of the sensors, the stimulating electrodes may issue an electric signal to directly assist CPR and/or to provide supportive counter-pressure via the one or more expandable and collapsible units, providing controlled dynamic assistance to the CPR treatment. To simplify the discussion that follows, an insertable expandable and collapsible unit may herein be referred to as “expandable unit”. The one or more expandable units may be operative to increase perfusion in the patient when in operable position. For example, after insertion into the patient via his/her esophagus, at least one of the expandable units may be positioned and retained in expanded configuration to harden or render a portion of the esophagus substantially non-compliant such to provide support to a posterior side of the heart during application of cardiac compression procedures and/or to subject the aorta to external pressure.

Cardiac compression procedures may include repetitively applying external cardiac compression procedures such as, for example, alternatingly exerting pressure on and releasing pressure from the patient's sternum. The procedure of exerting pressure on the patient's sternum may herein be referred to as “compression phase”, and the procedure of releasing pressure exerted on the patient's sternum may herein be referred to as “decompression phase”.

According to some embodiments, during the compression phase, the posterior side of the heart may be pushed against an anterior side portion of the esophagus. A portion of the esophagus, which is supported from the rear by the patient's vertebral column may be hardened or stiffened by the expanded unit inserted therein.

Accordingly, the magnitude of the pressure exerted on the cardiac chambers may be increased compared to the pressure that may be applied on the cardiac chambers if no expandable unit was present, which may result in increased compression of the cardiac chambers and, in turn, in correspondingly improved artificial blood circulation and increased perfusion of vital organs.

According to some embodiments, the CPR assisting apparatus may include one or more sensors operative to provide a feedback signal relevant to applying CPR, such as a pressure-related measurement corresponding to external pressure applied on the patient during external cardiac compression. Optionally, the sensors may provide additional information, such as temperature and/or a fluid-related measurement. For example, the sensors may measure the oxygen concentration, or air and/or blood pressure, and the like. Each of expansion and collapsing of the expandable unit or units may be controlled based on the information provided by the sensors, such as by using the feedback signal received from the sensor(s) to operate one or more actuators. The sensors may be operative to provide the feedback signals while positioned within and/or external to the patient's body. In some embodiments, the actuators may control the operation of the expandable units based on one or more vital sign-based measurements corresponding to an applied electrical defibrillation, pacing and/or gasping-stimulation. Alternatively, the actuators may control the operation of the expandable units in accordance with predetermined treatment program.

According to some embodiments, the CPR assisting apparatus may include a gasping trigger arrangement configured to apply a stimulus to the patient's thorax diaphragm that induces the patient to inhale. Additionally or alternatively, the gasping trigger arrangement may control one or more electrodes that selectively apply one or more electrical defibrillation or heart pacing signals. Such electrodes may herein be referred to as defibrillation/pacing electrodes.

The gasping trigger arrangement may include one or more electrodes that may be positioned at the patient's thorax diaphragm in a manner suitable for stimulating the thorax diaphragm to trigger gasping. The operation of gasping trigger arrangement may be controlled based on any suitable measurement or signal, such as based on the pressure-based or other information provided by any of the sensors, one or more monitored vital sign parameters, in accordance with the delivery of electrical defibrillation and/or pacing signals, and/or in accordance with the operation of the inserted expandable units; or according to a predetermined program.

Additionally or alternatively, gasping triggering arrangement may include an esophagus electrode (e.g., anode electrode) operably positionable within the patient's upper esophagus for example at about C6 level, e.g., on an expandable unit positionable in the esophagus at about C6 level. The arrangement may further include one or more neck electrodes (e.g., a bilateral neck cathode) operably positionable (external of the patient's body) at the posterior border of the sternocliedomastoid muscles at the cricoid cartilage level. When in operable position, a signal may be directed from the one or more neck electrodes to the esophageal electrode to stimulate the cervical right and/or left phrenic nerves. The magnitude of the electrical current may range, for example, from about 150 milliampere to about 250 milliampere, ±10%, or from about 200 milliampere to about 250 milliampere, ±10%.

According to some embodiments, delivery of electrical defibrillation or gasping energy may be based on the pressure-based output, values of monitored vital sign parameters, in accordance with the delivery of electrical gasping stimulation energy, and/or in accordance with the expansion and/or collapsing of the expandable units.

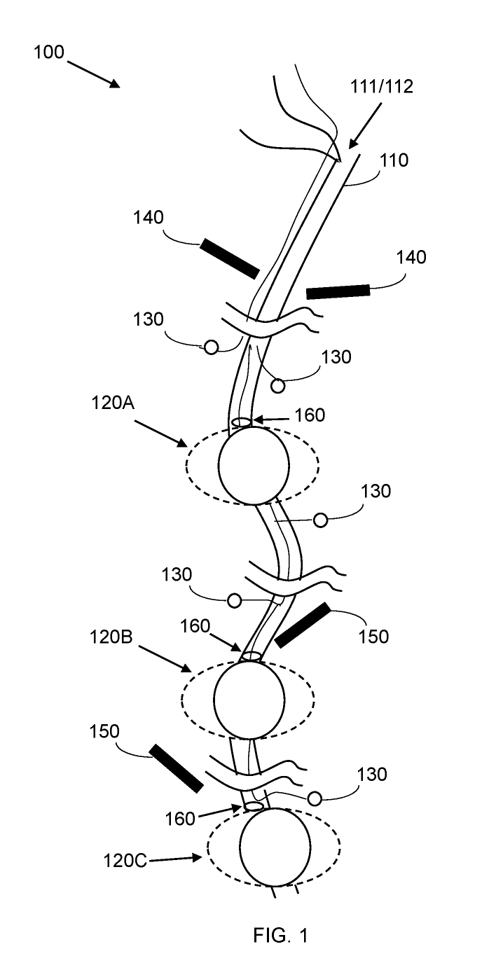

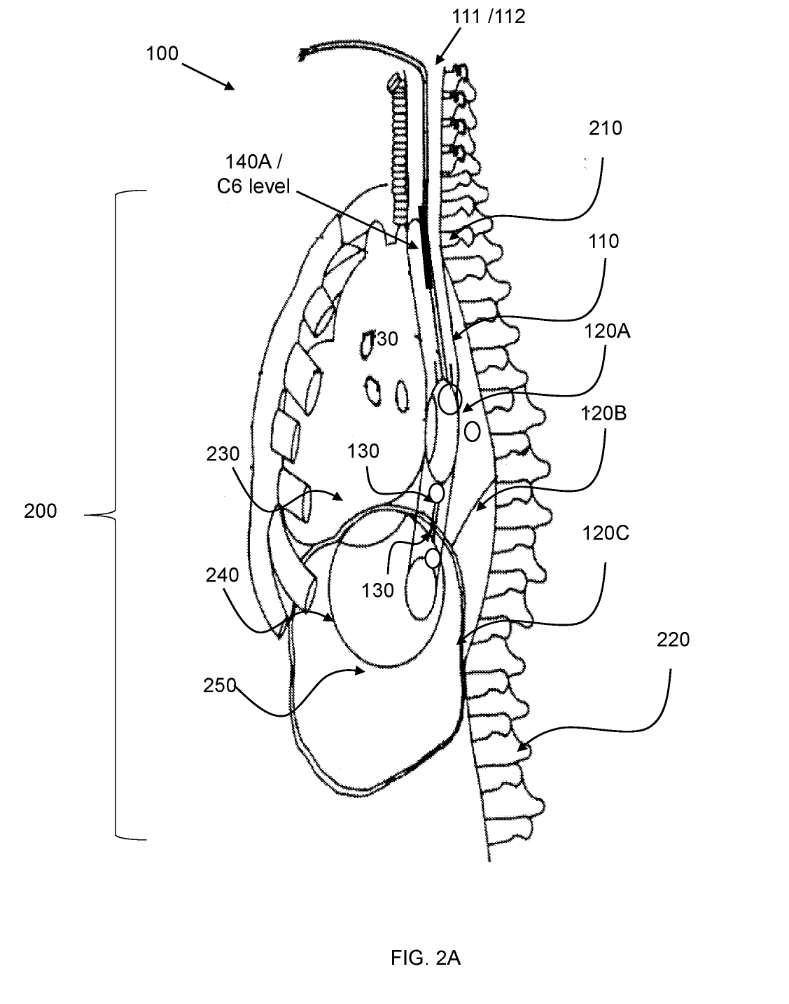

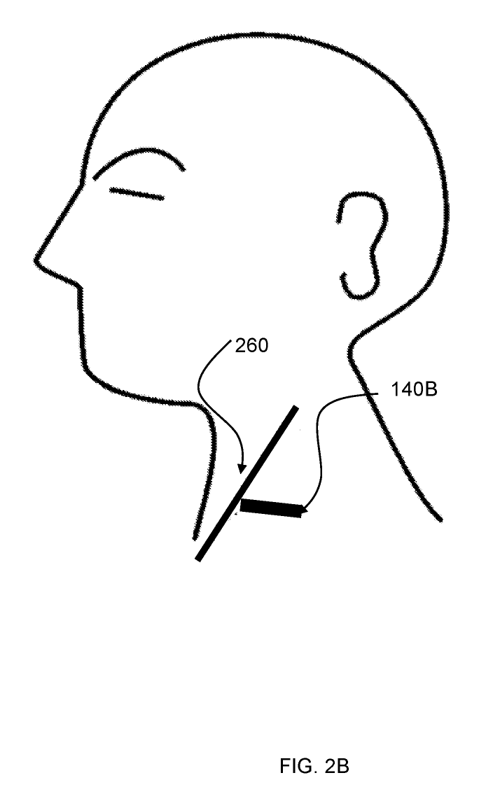

Reference is now made to FIGS. 1 and 2A-2B which, taken together, illustrate a controllable CPR assisting apparatus 100 that may be applied a chest cavity 200 of a patient, as outlined herein in greater detail. CPR assisting apparatus 100 may include an elongate body 110 and one or more expandable units 120 that are coupled with and arranged along elongate body 110. CPR assisting apparatus 100 may further include one or more actuators 160 for individually and/or collectively controlling the expansion and collapsing of units 120, such as in response to one or more measurements obtained by one or more sensors 130. For example, each of expandable units 120 may be configured with one of actuators 160 having a valve that independently controls the fluid flow in and out of each of units 120. Thus, actuators 160 may control the operation of the valves to allow independently expanding and/or collapsing each of units 120, such as via a controller which is described in greater detail below. At least a portion of elongate body 110 may be sufficiently flexible to be passed through the esophagus of the patient's human chest cavity 200.

The patient's esophagus is herein designated by alphanumeric reference “210”, the vertebral column is designated by alphanumeric reference “220”, the patient's heart is designated by alphanumeric reference “230”, the thorax diaphragm is designated by alphanumeric reference “240” and the stomach is designated by alphanumeric reference “250”.

Elongate body 110 may comprise a tube for providing a fluid passageway having an inlet 111 and outlet 112. Inlet 111 and outlet 112 are for mere simplicity illustrated as being identical or coinciding. However, this should by no means to be construed as limiting. Accordingly, in some embodiments, the fluid passageway, or tube, may have an inlet 111 that is separate from outlet 112. It is also noted that, in some embodiments, at least some of the passageway may include passageway portions that are separate from each other.

Tube 110 may be in fluid communication with expandable units 120. Tube 110 may allow a pressurized fluid to flow from inlet 111 to expandable units 120 and expand them accordingly. Similarly, tube 110 may allow the collapsing of expandable units 120 by releasing fluid through the fluid passageway via outlet 112.

After insertion of at least a portion of elongate body 110 and expandable units 120 into the patient via his/her esophagus 210 for setting CPR assisting apparatus 100 into its operable position, a first expandable unit 120A of the plurality of expandable and collapsible units 120 may be located proximal with respect to the patient's mouth and a second expandable unit 120B may located distally with respect to the patient's mouth. A third expandable unit 120C may be located distally to second expandable unit 120B.

Accordingly, after insertion and setting apparatus 100 in operable position, first expandable unit 120A may be positioned between second (distal) expandable unit 120B and the patient's mouth, and second expandable unit 120B is positioned between first unit 120A and third unit 120C.

According to some embodiments, when CPR assisting apparatus 100 is in operable position, as is for example shown schematically in FIG. 2A, any of expandable units 120A, 120B, and 120C may be expanded accordingly to provide support to esophagus 210 and/or the patient's inner stomach wall 250.

For example, first expandable unit 120A may be positioned within the patient's esophagus 210 between the patient's heart 230 and his/her vertebral column 220; second expandable unit 120B may be positioned within esophagus 210 at the height of the patient's descending aorta (not shown); and third expandable unit 120C may be positioned within the patient's stomach 250.

Accordingly, to simplify the discussion that follows and without being to be construed as limiting, first expandable unit may herein be referred to as “cardiac unit 120A”, second expandable unit may be referred to as “descending-aorta unit 120B”, and third expandable unit may be referred to as “gastric unit 120C”.

Cardiac unit 120A may be expanded and positioned relative to vertebral column 220 and heart 230 such to support a posterior side of the heart for maintaining in position at least some of the posterior side of heart 230 relative to the patient's vertebral column during application of cardiac compression procedures. For example, cardiac unit 120A may be positioned or sandwiched between vertebral column 220 and heart 230. To provide sufficient support, cardiac unit 120A, when set in expanded configuration, may be substantially non-compliant or rigid, at least with respect to applied external cardiac compression. Cardiac unit 120A may be filled with a substantially incompressible fluid (e.g., water). In some embodiments, compressible fluid may be employed including, for example, air and/or gas. When in operable position and set in expanded configuration, the anteroposterior dimension of cardiac unit 120A may decrease by not more than 1%, 2%, 3%, 4% or 5% during a compression phase of properly applied external cardiac compression.

Hence, by providing support of at least some of the posterior side of heart 230, the magnitude of the pressure exerted on one or more of the cardiac chambers may be increased compared to the pressure that may be applied on the cardiac chambers if cardiac unit 120A was not present. Generally speaking, an increase in pressure exerted on the cardiac chambers may result in increased compression thereof and, in turn, in correspondingly increased perfusion of vital organs. In some embodiments, the magnitude of pressure applied against a posterior of heart 230 may alternate by expanding and collapsing cardiac unit 120A.

In some embodiments, CPR assisting apparatus 100 may be operative such that cardiac unit 120A selectively causes increase and decrease of the pressure applied in one or more cardiac chambers of heart 230. It may for example be desirable that mainly or solely the heart's lower ventricles are compressed due to the application of external cardiac compression. Additional or alternative configurations are conceivable.

According to some embodiments, descending-aorta unit 120B of CPR assisting CPR assisting apparatus 100 may enable, when in operable position and set in expanded configuration, the application of pressure against a portion of the descending aorta (not shown) via esophagus 210. In some embodiments, application of pressure against the descending aorta and subsequent relaxation may be executed in an alternating manner by expanding and collapsing descending-aorta unit 120B.

During the compression phase, expandable units 120A and 120B may be in expanded configuration so that they may be subjected to intrathoracic pressure which may for example range from about 50 to 100 mmHg. This intrathoracic pressure may be transmitted by cardiac unit 120A to heart 230, and by descending-aorta unit 120B to the aorta.

During a decompression (or relaxation) phase, descending-aorta unit 120B may stay in expanded configuration at a pressure which may be limited by the strain-stress relation displayed by the esophagus. More specifically, pressure exerted on esophagus 210 by the expansion of unit 120B may be controlled to retain the elastic properties of the esophagus. Accordingly, pressure may be exerted against at least a portion of the patient's descending aorta during the employment of external cardiac compression.

It is noted that esophagus 210 may be able to withstand high pressure for relatively short durations of up to a few minutes. Expanding descending-aorta unit 120B may allow exerting pressure on the descending aorta to increase aortic diastolic pressure, resulting in a higher return venous pressure gradient than would otherwise be obtained in the absence of expanded unit 120, and which would result in a comparatively low coronary perfusion pressure. Thus, by applying pressure on the aorta via unit 120B, the obtained venous pressure gradient may be increased accordingly, and which, in turn, may increase coronary perfusion and probability of ROSC.

According to some embodiments, decompression may be implemented using an active compression-decompression pump (not shown). Active decompression may increase a venous return pressure gradient, which may be lower if “regular” or non-active decompression was employed instead.

According to some embodiments, gastric unit 120C may be positioned and expanded to provide support to the lower ventricle of heart 230, by pressing against the endothelial wall of stomach 250 located next to the left lower ventricle. This may for example be achieved by pulling of the elongate body in outward direction from esophagus 210. This procedure may also assist in positioning of expandable units 120 in their desired position, as outlined herein below in more detail.

According to some embodiments, CPR assisting apparatus 100 may be operative to enable the application of pressure against the inner stomach wall and subsequent relaxation in an alternating manner by expanding and collapsing gastric unit 120C.

Expandable units 120 may be sufficiently small when collapsed to allow insertion into the esophagus and subsequent removal. When expanded, units 120 may expand to a diameter that is sufficient to provide the functionalities described above. For example, descending-aorta unit 120B may expand to a diameter ranging between 3 to 3.5 centimeters (cm) ±10%, corresponding to a typical diameter of the aorta ranging from 2.5 to 3 cm, to allow exerting pressure on the aorta accordingly.

CPR assisting apparatus 100 may be operative such that expansion and collapsing of a first expandable unit occurs in coordination with expansion and/or collapsing of a second expandable unit, and/or in coordination with the external pressure applied, e.g., on the patient's sternum. Expandable units 120 may for example be operated in coordination such that during a compression phase, the cardiac blood discharge pressure is further increased, compared to the discharge pressure that would be attained if expandable units 120 were to retain in their expanded configuration during the compression and decompression phase.

Analogously, expandable units 120 (e.g., cardiac unit 120A) may be collapsed during the decompression phase to allow the return blood pressure into the right side of the patient's heart to increase more than the return blood pressure attainable if the units were to remain expanded during the compression and decompression phase.

The expanding and collapsing operation of units 120 may be coordinated by computerized CPR assist device 310 based on data received from sensors 130 or other suitable source, and/or based on the operation of a CPR, or active compression decompression pump.

CPR assisting apparatus 100 may be operative to allow for the positioning of expandable units 120 in their desired position in patient (e.g., along esophagus 210 and/or stomach 250), independent of the patient's height for example. In some embodiments, CPR assisting apparatus 100 may be operative to enable making use of one or more anatomy-related references. For example, gastric unit 120C may be inserted in collapsed configuration via esophagus 210 into the patient's stomach 250. Gastric unit 120C may then be expanded and forced against the top of the stomach wall, optionally until the stomach wall contacts the bottom of the heart. Hence, the position where gastric unit 120C abuts against the top of the stomach wall provides an indication or reference for determining the position of heart 230 within the patient along esophagus 210. The reference may therefore assist in determining the operable position of cardiac unit 120A to provide support against a posterior of heart 230 during the compression phase and/or of descending-aorta unit 120B to allow for the compression of the descending aorta.

According to some embodiments, sensors 130 may be employed for setting expandable units 120 in operable position within the patient's chest cavity 200. For example electrodes (e.g., defibrillation/pacing electrodes 150) may be employed for that purpose. For instance, sensors 130 may be placed within esophagus 210 at various heights along the longitudinal axis. Additionally or alternatively, sensors 130 may be arranged, on at least one of the various heights, to circumferentially engage the inner side wall of esophagus 210. The sensors' output may be indicative of whether there was a change in the positioning of esophagus 210.

According to some embodiments, CPR assisting apparatus 100 may include one or more sensors 130 for providing, when in operable position, pressure-related data indicative of pressure exerted the patient's sternum during cardiac compression procedures. Sensors 130 are operatively coupled with expandable units 120 such that based on the pressure-related data, expandable units 120 may be expanded or collapsed. Sensors 130 may be embodied, for example, by pressure sensors, accelerometers, electrodes (e.g., defibrillation/gasping electrodes 150) for detecting heart movement; and/or any other type of sensor which may be employed for providing pressure-related data. Some sensors 130 may be internal and some external to the patient's body when in operable position.

CPR assisting apparatus 100 may further include a gasping trigger arrangement (not shown) which may for example comprise one or more gasping-stimulation electrodes 140. When in operable position, gasping-stimulation electrodes 140 may be operative to stimulate thorax diaphragm 240 and/or one of or two phrenic nerves, such to trigger gasping of the patient. The stimulation may be executed by electrodes that are internal and/or external to the patient.

Further referring to FIG. 2B, a left phrenic nerve, which is for illustrative purposes only schematically illustrated as a straight line and designated with alphanumeric label “260”, may be stimulated by employing esophagus electrode (e.g., anode electrode) 140A operably positionable within the patient's upper esophagus for example approximately at C6 level, e.g., on an expandable unit correspondingly positionable in the esophagus. The arrangement may further include one or more external neck electrodes 140B (e.g., a bilateral neck cathode) operably positionable (external of the patient's body) at the posterior border of the sternocliedomastoid muscles at the cricoid cartilage level. When in operable position, electrical energy can be directed from the one or more neck electrodes to the esophageal electrode at a magnitude suitable for stimulating the cervical right and/or left phrenic nerves. The magnitude of the electrical current may range, for example, from about 150 milliampere to about 250 milliampere, or from about 200 milliampere to about 250 milliampere.

CPR assisting apparatus 100 may further include defibrillation/pacing electrodes 150 which may additionally or alternatively be employed, when in operable position, for the delivery of internal and/or external-to-external electrical energy for defibrillation in case of shockable rhythm, or as pacing electrodes. Electrical pacing may be employed in case of bradycardia and during asystole. Gasping-stimulating electrodes 140 and/or defibrillation/pacing electrodes 150 may be set in their operable position (e.g., in the esophagus) in a minimally invasive manner or through intubation to allow internal delivery of electrical energy for gasping stimulation, defibrillation and/or pacing.

Operation of expandable units 120, the gasping trigger arrangement, and/or of the application of internal defibrillation or pacing may for example occur in coordination with each other and/or in accordance with the pressure-related output provided by sensors 130, as will be outlined herein below in greater detail.

It is note that in some embodiments, a gasping-stimulating electrode 140 may additionally or alternatively function as a defibrillation electrode or pacing electrode. Analogously, a defibrillation/pacing electrode 150 may additionally or alternatively function as a gasping-stimulating electrode. For example, a gasping-stimulating electrode may be arranged on gastric expandable unit 120C which may be forcefully abutted against the gastric fundus and cardia so that the latter presses against the diaphragm dome to apply, in turn, pressure against the basal aspect of heart 240. Thus configured, electrical defibrillation energy may pass between a gastric—cardiac and/or descending aorta electrodes route, and electrical gasping stimulation energy may pass between a gastric—posterior/lateral chest wall route.

The electrical pulsing rate delivered from a gasping electrode 140 may for example range between 4 to 6 pulses per minute and/or actuated according to operator's discretion, while defibrillation may for example be employed every two minutes and/or actuated according to clinician discretion, such as described in Algorithms for Advanced Cardiac Life Support (ACLS) protocols (available at: https://www.acls.net/aclsalg.htm, last seen Mar. 7, 2016).7

According to some embodiments, the electrical energy delivered for defibrillation from the electrodes, which may optionally be internal defibrillation, may for example be about 20 Joule ±10%.

According to some embodiments, a rate of electrical energy delivery for cardiac pacing may for example range from about 80 to about 100 electrical pulses per minute ±10%. The current provided at each pulse for pacing may for example range from about 5 to about 30 milliamperes, ±10%.

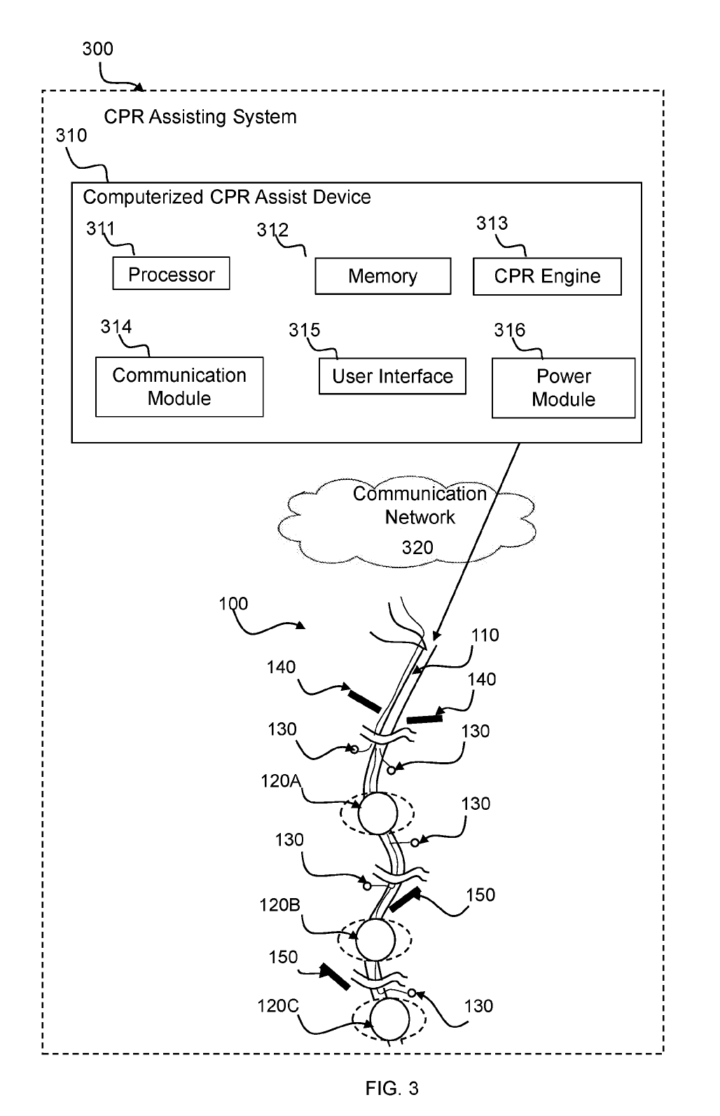

Further reference is made to FIG. 3. A system 300 or assisting an operating in performing CPR (hereinafter: CPR assisting system) may include a computerized CPR assist device 310 for operating CPR assisting apparatus 100. Components of CPR assisting 310 may be operatively coupled with components of CPR assisting apparatus 100 using wired and/or wireless communication (e.g., communication network 320).

CPR assist device 310 may for example be operative to control the expansion and collapsing of expandable units 120, the powering of gasping-stimulating electrodes 140 and/or of defibrillation electrodes 150.

CPR assist device 310 may include a processor 311, a memory 312, a communication module 314, a user interface 315 (which may comprise and input device and/or an output device) and a power module 316 for powering the various components of computerized CPR assist device 310 and/or CPR assisting apparatus 100. Components of computerized CPR assist device 310 may communicate with each other over one or more signal lines (not shown).

The terms “processor” as used herein may additionally or alternatively refer to a controller. Processor 311 may include and/or relate to various types of processors and/or processor architectures including, for example, embedded processors and/or communication processors.

According to some embodiments, memory 312 may include one or more types of computer-readable storage media including, for example, transactional memory and/or long-term storage memory facilities and may function as file storage, document storage, program storage, or as a working memory.

According to some embodiments, components or parts of components of computerized CPR assist device 310 such as, for example, processor 311, memory 312, communication module 314, user interface 315 and/or power module 316 may be shared and/or integrated with CPR assist apparatus 100.

In some embodiments, communication module 314 may communicate with CPR assist apparatus 100 over communication network 320 which may relate to a variety of networks and/or standards. Accordingly, CPR assist apparatus 100 may in some embodiments include a communication module (not shown), a processor (not shown) and/or a memory (not shown). Communication module 314 may for example include I/O device drivers (not shown) and network interface drivers (not shown). A device driver may for example, interface with a keypad or to a USB port. A network interface driver may for example protocols for short range wireless communication protocols like, for instance, Zigbee™, Bluetooth® and/or Wi-Fi protocols, the Internet, or an Intranet, Wide Area Network (WAN), Local Area Network (LAN) employing, e.g., Wireless Local Area Network (WLAN)), Metropolitan Area Network (MAN), Personal Area Network (PAN), extranet, 2G, 3G, 3.5G, 4G including for example Mobile WIMAX or Long Term Evolution (LTE) advanced, and/or any other current or future communication network, standard, and/or system.

While the components and/or features of CPR assisting system 300 are schematically illustrated as being implemented at a single location, this should by no means to be construed limiting. Accordingly, elements of any one of the components of CPR assisting system 300 may be implemented at various locations, e.g., of CPR assisting apparatus 100 and/or computerized CPR assist device 310. For example, some parts of memory 312 may be included in sensors 130 and/or in any other element of the wearable electronic device.

Processor 311 may be operative to run or execute one or more sets of instructions stored in memory 312 to result in a CPR assisting engine 313 for controlling operation of components of CPR assisting apparatus 100.

Power module 316 may comprise a power source embedded in Computerized CPR assist device 310 which may be operatively coupled (e.g., through a USB connection or any other suitable wired or wireless connection) with components of CPR assisting apparatus 100 including, for example, actuators 160 for actuating the expansion and collapsing of expandable units 120, sensors 130, gasping-stimulation electrodes 140 and/or defibrillation/pacing electrodes 150. Additionally or alternatively, power module 316 may enable connecting CPR assist device 110 to an external power source (not shown).

According to some embodiments, CPR assisting engine 313 may cause expansion and collapsing of expandable units 120 through operation of their corresponding actuators 160 based on pressure-related data provided by sensors 130, values of vital signs parameters, operation of the CPR pump and/or in accordance with electrical defibrillation and/or gasping stimulation energy delivered. Monitored vital signs may relate, for example, to electrical cardiac activity indications obtained via, e.g., defibrillation/pacing electrodes 150 which may thus, in some embodiments, function as sensors 130.

A CPR engine 313 may be provided to control the expansion and/or contraction of any of units 120A, 120B, and 120C. For example, CPR engine 313 may identify the occurrence of a compression phase and, in response, cause expandable cardiac unit 120A to be in expanded configuration and/or cause descending-aorta unit 120B to be in a collapsed configuration during the compression phase by operating the respective actuators 160 accordingly.

Additionally or alternatively, CPR engine 313 may further be configured to identify the occurrence of a decompression phase and, in response, cause expandable cardiac unit 120A to be in collapsed configuration through suitable actuator operation and/or cause descending-aorta unit 120B to be in an expanded configuration by operating the respective actuators 160 accordingly.

Additionally or alternatively, gastric unit 120C may be set into expanded or collapsed configuration in accordance with the cardiac compression phase applied. For instance, expandable gastric unit 120C may be set to be in expanded configuration by CPR engine 313 during the compression phase, and in collapsed configuration during the decompression phase.

Correspondingly, CPR engine 313 may be operative to cause expandable cardiac unit 120A, expandable descending-aorta unit 120B and/or gastric unit 120C to alternate between a collapsed and expanded configuration, for example, in accordance with the applied cardiac compression.

At some instances, during the compression phase, CPR engine 313 may cause cardiac unit 120A and gastric unit 120C to be in collapsed configuration and descending-aorta unit 120B to be in expanded configuration, and vice versa.

Additional or alternative operating schemes may be implementable by CPR assisting apparatus 100. For example, CPR engine 313 may cause cardiac unit 120A, descending-aorta unit 120B and/or gastric unit 120C to be set in a collapsed configuration during a first set of compression and decompression phases and in expanded configuration during a second set of compression and decompression phases which is followed by the first set. The number of instances of compression and decompression phases in the first and second set may equal or differ from each other. For example, each one of a first and second set of decompression phases may include two or more compression and decompression phases. In another example, a first set of compression and decompression phases may include three compression and two decompression phases, whereas a second set may include correspondingly alternating five decompression and four compression phases. In some embodiments, a set of phases may include either a single compression or a single decompression phase.

According to some embodiments, a CPR sequence may be as follows: electrical gasping stimulation energy may be delivered, e.g., for a few seconds, followed by external cardiac compression procedure for about, e.g., 2 minutes. Following external cardiac compression procedure, electrical defibrillation energy may be delivered, e.g., for a few seconds. If Return of Spontaneous Circulation (ROSC) occurs, which may for example be indicated by an electrocardiographic (ECG) signal showing organized electrical activity, from pressure-related data indicative of pressure above perfusion-threshold and/or palpable pulse following defibrillation for example, CPR procedure may be halted and any of units 120A, 120B, and 120C may be deflated accordingly, responsive to the ROSC indication. Otherwise, the CPR sequence may be repeated. In some embodiments, gasping stimulation may be applied via gasping-stimulating electrode 140 during the employment of the CPR procedure (e.g., during external cardiac compression). The gasping stimulation may be delivered without delivering defibrillation energy to heart 230.

According to some embodiments, pressure-related data received for example from sensors 130 may serve as a quality indication of the external cardiac compression employed, e.g., based on compression depth, compression rate and/or level of relaxation which may be derivable from the pressure-related data.

According to some embodiments, CPR engine 313 may provide one or more activating signals to gasping-stimulating electrodes 140 and/or defibrillation/pacing electrodes 150 for stimulating gasping in the patient and/or for defibrillating the patient's heart, e.g., based on pressure-related data received from sensors 130. For example, CPR engine 313 may activate gasping-stimulation electrodes 140 and/or defibrillation/pacing electrodes 150 responsive to receiving one or more physiological indicators, such as pressure-related measurements, from sensors 130 indicating little or no externally applied cardiac compression. For instance, when the pressure-related data received from sensors 130 indicates that CPR is in the decompression phase, CPR engine 313 may activate gasping-stimulating electrodes 140 and/or defibrillation/pacing electrodes 150.

According to some embodiments, defibrillation/gasping electrodes and/or pacing electrodes 140 and 150 may be selectively activated based on one or more operating parameters, such as data received from sensors 130, the operation of a CPR pump; and/or based on values of monitored vital signs parameters.

According to some embodiments, CPR engine 313 may indicate via user interface 315 of computerized CPR assist device 310 that gasping stimulation and/or defibrillation is about to be initiated, for example, in order to warn an operator of CPR assisting system 300 to not touch the patient to avoid subjecting the operator to electrical shock.

According to some embodiments, CPR engine 313 may adjust the duration and/or magnitude of electrical energy to be provided via gasping-stimulating electrodes 140 and/or defibrillation/pacing electrodes 150, e.g., based on the pressure-related data received from sensors 130 and/or data descriptive of values of vital signs parameters such as, for example, values indicative of electrical cardiac activity obtained, e.g., via defibrillation electrodes 150 of computerized CPR assist device 310.

For example, if thoracic pressure as measured by sensors 130 exceeds a certain threshold, descending-aorta unit 120B may be expanded and no defibrillation energy may be delivered. If the measured pressure drops below a certain threshold value and shockable rhythm, such as ventricular fibrillation or pulseless ventricular tachycardia, is detected by CPR engine 313 via one or more signals received from the one or more defibrillation/pacing electrode 150, a signal to activate electrical defibrillation may be provided, followed by a signal to activate gasping stimulation, such as after electrical defibrillation energy drops substantially to zero. In some embodiments, if the measured thoracic pressure drops below a threshold value, and a non-shockable rhythm, such as asystole or pulseless electrical activity, is detected by CPR engine 313 via one or more signals received from the one or more defibrillation/pacing electrode 150, a signal to activate gasping-stimulation may be delivered. During non-shockable periods, such as during bradycardia and asystole, CPR engine 313 may activate defibrillation/pacing electrodes 150. According to some embodiments, electrical defibrillation and/or gasping-stimulation signals may be delivered if at least one criterion relating to the application of external pressure is met. For example, signals may be delivered if the number of events of external pressure application within a period of time is above an event-count threshold. Additionally or alternatively, the at least one criterion may comprise a condition for the delivery of electric signals when applied pressure exceeds a certain minimal-pressure threshold. For example, the minimal-pressure threshold may for example relate to the magnitude of the pressure for each instance of successively applied external cardiac compression, optionally within a period of time. Additionally or alternatively, defibrillation and/or gasping stimulation signals may be delivered when accumulated pressure applied within, e.g., a capped number of successive external cardiac compression, optionally within a period of time, exceeds a certain pressure-threshold.

According to some embodiments, defibrillation and/or gasping-stimulating signals may be delivered when values of patient vital signs may be below a perfusion-threshold.

For example, electrical defibrillation signals may be provided via defibrillation electrodes 150 at higher rates the more the values of vital signs parameters are indicative of increased risk of terminal patient system failure, and vice versa. In some embodiments, CPR engine 313 may adjust the magnitude of the electrical defibrillation signals and/or gasping-stimulation signals in accordance with measured values of vital signs parameters.

According to some embodiments, procedures for gasping stimulation may be initiated by CPR engine 313 via gasping-stimulation electrodes 140, e.g., when substantially no defibrillation energy is delivered via defibrillation/pacing electrodes 150. Correspondingly, defibrillation procedures may be initiated by CPR engine 313 via defibrillation/pacing electrodes, e.g., when substantially no electrical defibrillation energy is fed into the patient via gasping-stimulating electrodes 140.

According to some embodiments, at least for some period of time, gasping-stimulating electrodes 140 may be activated concurrently with defibrillation/pacing electrodes 150.

According to some embodiments, CPR engine 313 may provide its operator via user interface 315 with guidance on how to execute cardiac resuscitation procedures.

In some embodiments, the timing of expansion and/or collapsing of expandable units 120 and/or the timing for providing electrical defibrillation and/or gasping-stimulation energy may occur according to a predetermined schedule, hereinafter referred to as “first operating mode”.

According to some embodiments, CPR engine 313 may switch from the first operating mode in which expandable units 120 and/or electrical defibrillation and/or gasping-stimulation energy are operated according to a predetermined schedule, to a second operating mode, in which actuators 160 of expandable units 120 are operated and/or energy for defibrillation and/or electrical-gasping stimulation are provided, e.g., based on pressure-related data received from sensors 130 and/or based on values of monitored vital signs parameters.

For example, after initiating operation of computerized CPR assist device 310, CPR engine 313 may by default operate actuators 160 in the first operating mode. After a certain period of time, e.g., depending on values of monitored vital sign parameters and/or pressure-based data, CPR engine 313 may switch from the first to the second mode of operation.

According to some embodiments, CPR engine 313 may expand any of expandable units 120 in a manner to maximize blood flow to one or more vital organs such as the brain and heart. For example, expandable units 120 may be expanded in conjunction with the expansion of additional expandable units inserted into the femoral artery (not shown), to restrict blood flow to the lower body and increase blood flow to the vital organs.

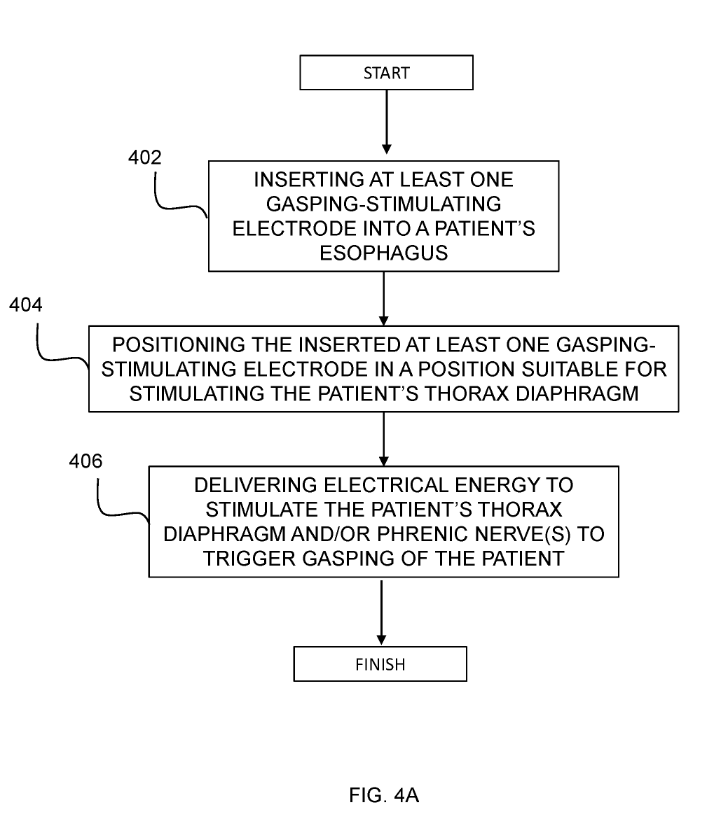

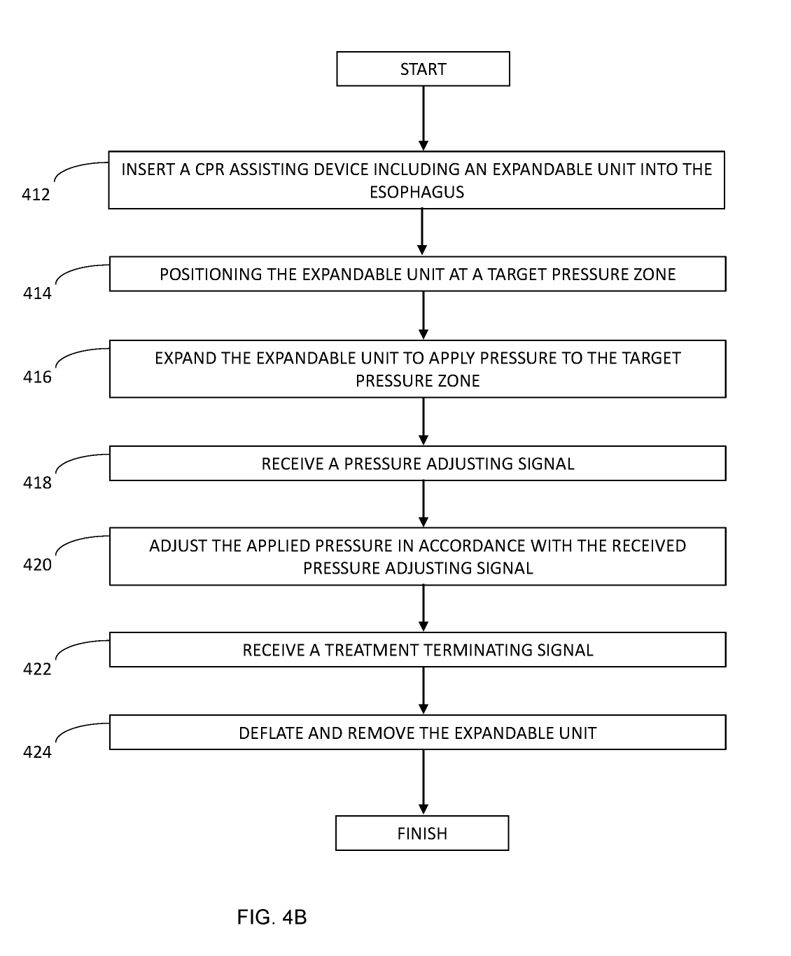

Additional reference is made to FIG. 4A. As indicated by box 402, a method for assisting in the treatment of cardiac arrest in a patient may include inserting a gasping-stimulating electrode into a patient's esophagus. As indicated by box 404, the method may include positioning the inserted gasping-stimulating electrode to stimulate the patient's thorax diaphragm and cause a gasping action by the patient. As indicated by box 406, the method may include delivering a signal to the gasping-stimulating electrode that stimulates the patient's thorax diaphragm and/or phrenic nerve(s) 260 to cause a gasping action by the patient. Optionally, the signal may be controlled by CPR engine 313.

Additional reference is made to FIG. 4B which illustrates a method for assisting an application of CPR. A device for assisting CPR application including at least one expandable unit may be inserted into the esophagus (Step 412). The expandable unit may be positioned at a target pressure zone, such as in proximity to the descending aorta (Step 414). The expandable unit may be expanded at the target pressure zone, to apply pressure to the target pressure zone (Step 416). For example, pressure may be applied against at least a portion of the patient's descending aorta, such as when applying external cardiac compression. One or more pressure adjusting signals such as may relate to a measurement received from a sensor configured with the device, or an external measurement, or other indication may be received (Step 418). The pressure in the expandable unit may be adjusted in accordance with the pressure adjusting signal (Step 420). Steps 412 to 420 may be repeated for multiple expandable units. A signal to terminate the operation of the device may be received (Step 422). The expandable unit may be deflated and removed from the esophagus (Step 424). It should be noted that the expression “mounted on” may also encompass the meaning of the expression “mounted in” or “included in”.

The terms “right”, “left”, “bottom”, “below”, “lowered”, “lower”, “low”, “top”, “above”, “upper”, “elevated” and “high” as well as grammatical variations thereof as used herein do not necessarily indicate that, for example, a “lower” component is below an “upper” component, or that a component that is “lower” is indeed “below” another component or that a component that is “upper” is indeed “above” the “lower” component as such directions, components or both may be flipped, rotated, moved in space, placed in a diagonal orientation or position, placed horizontally or vertically, or similarly modified. Accordingly, it will be appreciated that the terms “lower”, “bottom”, “below”, “upper”, “top” and “above” may be used herein for exemplary purposes only, to illustrate the relative positioning or placement of certain components, to indicate a first and a second component or to do both.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

Unless otherwise stated, the use of the expression “and/or” between the last two units of a list of options for selection indicates that any selection of one or more of the listed options is appropriate and may be made.

It should be understood that where the claims or specification refer to “a” or “an” element, such reference is not to be construed as there being only one of that element.

The various features and steps discussed above, as well as other known equivalents for each such feature or step, can be mixed and matched by one of ordinary skill in this art to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein. For example, any digital computer system (exemplified herein as CPR Assisting System 300) can be configured or otherwise programmed to implement a method disclosed herein, and to the extent that a particular digital computer system is configured to implement such a method, it is within the scope and spirit of the disclosure. Once a digital computer system is programmed to perform particular functions pursuant to computer-executable instructions from program software that implements a method disclosed herein, it in effect becomes a special purpose computer particular to an embodiment of the method disclosed herein. The techniques necessary to achieve this are well known to those skilled in the art and thus are not further described herein.

Computer executable instructions implementing an embodiment of a method disclosed herein can be distributed to users on a non-transitory computer-readable medium and are often copied onto a hard disk or other storage medium. When such a program of instructions is to be executed, it is usually loaded into the random access memory of the computer, thereby configuring the computer to act in accordance with a method disclosed herein. All these operations are well known to those skilled in the art and thus are not further described herein. The term “computer-readable medium” encompasses distribution media, intermediate storage media, execution memory of a computer, and any other medium or device capable of storing for later reading by a computer a computer program implementing embodiments of a method disclosed herein.

While this disclosure has been described in terms of certain embodiments and generally associated methods, alterations and permutations of the embodiments and methods will be apparent to those skilled in the art. The disclosure is to be understood as not limited by the specific embodiments described herein, but only by the scope of the appended claims.

1. A cardiopulmonary resuscitation (CPR) assisting apparatus, comprising:
a gasping-stimulating electrode configured to stimulate the thorax diaphragm to cause a gasping action; and
a controller configured to control the stimulation applied by the gasping-stimulating electrode.

2. The CPR assisting apparatus of claim 1, further comprising a sensor configured to measure a value relating to a physiological parameter, wherein the controller is configured to control the stimulation responsive to receiving the measured value.

3. The CPR assisting apparatus of claim 2, wherein the physiological parameter is an externally applied cardiac compression.

4. The CPR assisting apparatus of claim 1, wherein the gasping-stimulating electrode comprises:
an esophagus electrode configured to be positioned internal to the esophagus at the C6 vertebrate level; and a neck electrode configured to be positioned externally at a posterior border of the sternocliedomastoid muscles at the cricoid cartilage level,
wherein the neck electrode is configured to transmit to the esophagus electrode a stimulating signal suitable for stimulating any of a cervical right and left phrenic nerves.

5. The CPR assisting apparatus of claim 1, further comprising: at least one expandable unit configured to be inserted into the esophagus; and an elongated body in fluid communication with the expandable unit and configured to allow a pressurized fluid to flow to and from the expandable unit, thereby allowing expandable unit to expand and contract, wherein the controller is configured to control the expansion and contraction of the expandable unit.

6. The CPR assisting apparatus of claim 5, wherein the expandable unit comprises an expandable cardiac unit that is configured to render a portion of the esophagus substantially non-compliant.

7. The CPR assisting apparatus of claim 5, wherein the expandable unit comprises an expandable descending-aorta unit that is configured to apply pressure on the descending aorta.

8. (canceled)

9. The CPR assisting apparatus of claim 2, wherein the sensor comprises a defibrillation/pacing electrode, wherein the controller is configured to control a defibrillating stimulus delivered to the heart by the defibrillation/pacing electrode.

10. (canceled)

11. A cardiopulmonary resuscitation (CPR) assisting apparatus, comprising:
at least one expandable unit configured to be inserted into the esophagus;
an elongated body in fluid communication with the expandable unit and configured to allow a pressurized fluid to flow to and from the expandable unit, thereby allowing expandable unit to expand and contract; and
a controller configured to control the expansion and contraction of the expandable unit.

12. The CPR assisting apparatus of claim 11, wherein the expandable unit comprises an expandable descending-aorta unit that is configured to apply pressure on the descending aorta for increasing a venous return pressure gradient.

13. (canceled)

14. The CPR assisting apparatus of claim 11, wherein the expandable unit comprises an expandable gastric unit is configured to apply pressure on a portion of the inner wall of the stomach.

15. The CPR assisting apparatus of claim 11, further comprising a sensor configured to measure a physiological indicator and provide the measured indicator to the controller,
wherein the controller is configured to control the expansion and contraction responsive to receiving the measured indicator.

16. The CPR assisting apparatus of claim 15, wherein the physiological indicator is an externally applied cardiac compression.

17. A method for assisting in the treatment of cardiac arrest, the method comprising:
inserting at least one gasping-stimulating electrode into an esophagus;
positioning the gasping-stimulating electrode in a position suitable for stimulating the thorax diaphragm; and
delivering a signal to stimulate the thorax diaphragm to cause a gasping action.

18. (canceled)

19. A method for assisting in the treatment of cardiac arrest, the method comprising:
inserting a first expandable unit into the esophagus;
positioning the first expandable unit against at least a portion of the descending aorta; and
expanding the first expandable unit to apply pressure to the descending aorta during the application of external cardiac compression.

20. The method of claim 19, further comprising:
inserting a second expandable unit into the esophagus;
positioning the second expandable to provide support to a posterior side of the heart; and
expanding the second expandable unit to harden a portion of the esophagus, thereby providing support posterior side of the heart.

21. (canceled)

22. A cardiopulmonary resuscitation (CPR) assisting apparatus, comprising:
at least two expandable units configured to be inserted into the esophagus;
an elongated body in fluid communication with the expandable units and configured to allow a pressurized fluid to independently flow to and from each of the expandable units, thereby allowing each expandable unit to independently expand and contract; and
a controller configured to control the independent expansion and contraction of each of the expandable units.

23. The CPR assisting apparatus of claim 22, wherein the at least two expandable units comprise any two of: an expandable descending-aorta unit that is configured to apply pressure on the descending aorta for increasing a venous return pressure gradient,
an expandable cardiac unit that is configured to render a portion of the esophagus substantially non-compliant and support to a posterior side of the heart during application of cardiac compression procedures, and expandable gastric unit is configured to apply pressure on a portion of the inner wall of the stomach.

24. (canceled)

25. The CPR assisting apparatus of claim 22, further comprising a sensor configured to measure a physiological indicator and provide the measured indicator to the controller;
wherein the controller is configured to control the independent expansion and contraction of each of the expandable units responsive to receiving the measured indicator.

26. The CPR assisting apparatus of claim 22, wherein the physiological indicator is an externally applied cardiac compression.

27. (canceled)
